(19) United States
(12) Patent Application Publication
(10) Pub. No.: US 2023/0002468 A1
(43) Pub. Date: Jan. 5, 2023

(54) N-TERMINAL EXTENSION SEQUENCE FOR EXPRESSION OF RECOMBINANT THERAPEUTIC PEPTIDES

(71) Applicant: Biological E Limited, Hyderabad, Telangana (IN)

(72) Inventors: Ramesh Venkat MATUR, Hyderabad, Telangana (IN); Rajan SRIRAMAN, Hyderabad, Telangana (IN); Pavan Reddy REGATTI, Hyderabad, Telangana (IN); Narender Dev MANTENA, Hyderabad, Telangana (IN); Mahima DATLA, Hyderabad, Telangana (IN)

(21) Appl. No.: 17/763,785

(22) PCT Filed: Sep. 12, 2020
(86) PCT No.: PCT/IN2020/050790

§ 371 Date: Mar. 25, 2022

(30) Foreign Application Priority Data
Sep. 13, 2019 (IN) 201941009728

(51) Int. Cl. C07K 14/605; C12N 15/81; C12N 15/77; C12N 15/70; C12N 15/75; C12P 21/02

(52) U.S. Cl. CPC C07K 14/605; C12N 15/81; C12N 15/77; C12N 15/70; C12N 15/75; C12P 21/02; C07K 2319/50; C07K 2319/02

(57) ABSTRACT

The invention relates to an N-terminal extension sequences which are employed to enhance the expression of recombinant therapeutic peptides. The invention also relates to a process for the high-level expression of recombinant therapeutic peptides using the said N-terminal extension sequence. The invention also provides nucleic acids, vectors and recombinant host cells for efficient production of biologically active proteins such as lirapeptide.

### FIELD OF THE INVENTION

The invention relates to an N-terminal extension sequence for high-level expression of recombinant therapeutic peptides. The invention also relates to a process for high-level expression of recombinant therapeutic peptides using the said N-terminal extension sequence.

### BACKGROUND OF THE INVENTION

Peptide therapeutics have played a notable role in medical practice since the advent of insulin therapy in the 1920s. Currently, there are more than 60 approved peptide drugs in the market, and the numbers are expected to grow significantly.

Glucagon-like peptide-1 (GLP-1) is a 31 amino acid long peptide hormone deriving from the tissue-specific post-translational processing of the proglucagon peptide. It is produced and secreted by intestinal enteroendocrine L-cells and certain neurons within the nucleus of the solitary tract in the brainstem upon food consumption. Liraglutide is a derivative of a human incretin (metabolic hormone), glucagon-like peptide-1 (GLP-1) that is used as a long-acting glucagon-like peptide-1 receptor agonist, binding to the same receptors as the endogenous metabolic hormone GLP-1 that stimulates insulin secretion.

Teriparatide is a recombinant protein form of parathyroid hormone consisting of the first (N-terminus) 34 amino acids, which is the bioactive portion of the hormone. It is an effective anabolic (promoting bone formation) agent used in the treatment of some forms of osteoporosis.

An expression plasmid is engineered to contain regulatory sequences that act as enhancer and promoter regions and lead to efficient transcription of the gene carried on the expression vector. The goal of a well-designed expression vector is the efficient production of protein, and this may be achieved by synthesizing a significant amount of stable messenger RNA.

It is possible to design expression vectors that exert a tight control of the expression, and the protein is only produced in high quantity when necessary through the use of suitable expression condition. In absence of the tight control of the gene expression, the protein may also be expressed constitutively.

U.S. Pat. No. 4,916,212 discloses DNA-sequence encoding biosynthetic insulin precursors and the process for preparing the insulin precursors and human insulin in a yeast cell.

U.S. Pat. No. 7,572,884 discloses a method for preparing recombinant Lirapeptide, a precursor of Liraglutide in Saccharomyces cerevisiae.

IN 201741024763 A discloses a process for the preparation of Liraglutide by expression of synthetic oligonucleotide encoding Lirapeptide which is operably connected to an oligonucleotide sequence of a signal peptide in a yeast cell.

WO 1998/008871 A1 discloses derivatives of GLP-1 and analogues thereof prepared using recombinant DNA technique.

WO 1998/008872 A1 discloses derivatives of GLP-2 prepared using recombinant DNA technique.

WO 1999/043708 A1 discloses derivatives exendin and of GLP-1(7-C), prepared using recombinant DNA technique.

WO 2017/021819 A1 discloses a process for the preparation of peptides or proteins or derivatives thereof by expression of synthetic oligonucleotide encoding desired protein or peptide in a prokaryotic cell as ubiquitin fusion construct.

Avicenna J Med Biotech 2017; 9(1): 19-22 discloses overexpression of teriparatide (1-34), a recombinant bioactive part of human parathyroid hormone (PTH) in Escherichia coli.

The inventors of the present invention, in their endeavour to enhance the expression of the recombinant therapeutic peptides by several folds, have come up with the use of a short N-terminal extension sequence which is not disclosed in the above mentioned prior art.

### OBJECTIVE OF THE INVENTION

It is an objective of the present invention to provide high-level expression of the therapeutic peptides by several folds.

### SUMMARY OF THE INVENTION

The present invention provides N-terminal extensions, nucleic acids, vectors and recombinant host cells for efficient production of biologically active peptides such as lirapeptide.

The invention contemplates a multidimensional approach for achieving a high yield of peptides such as lirapeptide in a host cell by providing an expression construct in which the nucleic acid encoding lirapeptide is operably fused to a modified gene sequence encoding TEV (Tobacco Etch Virus) cleavage site and an N-terminal extension (NE-3).

The present invention provides an N-terminal extension sequence as set forth in SEQ ID NO: 1 (NE-3) to enhance the expression of a therapeutic peptide in bacteria or yeast.

The present invention also provides expression vectors and recombinant host cells for high-level expression of Lirapeptide, wherein the expression vector comprises a modified gene sequence encoding the N-terminal extension sequence NE-3, a modified gene sequence encoding TEV (Tobacco Etch Virus) cleavage site, and a modified gene sequence encoding Lirapeptide.

### BRIEF DESCRIPTION OF THE DRAWINGS

FIG. 1A: Schematic diagram of Expression cassettes without N-terminal extension.

FIG. 1 B: Schematic diagram of Expression cassettes with N-terminal extension (NE1).

FIG. 1 C: Schematic diagram of Expression cassettes with N-terminal extension (NE3).

FIG. 2A: Expression plasmid without N-terminal extension.

FIG. 2B: Expression plasmid with N-terminal extension-1 (NE1).

FIG. 2C: Expression plasmid with N-terminal extension-3 (NE3).

FIG. 3: Lirapeptide expression by ELISA.

FIG. 4: Dry cell weight of Lirapeptide.

### DESCRIPTION OF SEQUENCE LISTING

SEQ ID NO: 1 (amino acid sequence of N-terminal extension sequence NE-3)
EEQAE
SEQ ID NO: 2 (amino acid sequence of the modified TEV cleavage site)
ENLYFQ
SEQ ID NO: 3 (amino acid sequence of Lirapeptide)
HAEGTFTSDVSSYLEGQAAKEFIAWLVRGRG
SEQ ID NO: 4 (nucleic acid sequence encoding the N-terminal extension NE-3 sequence
for Pichia pastoris)
gaagaacaagccgaa
SEQ ID NO: 5 (nucleic acid sequence encoding the modified TEV cleavage site for Pichia
pastoris)
gagaacttgtacttccaa
SEQ ID NO: 6 (nucleic acid sequence encoding the lirapeptide for Pichia pastoris)
cacgctgagggtacttttacctctgacgtgtcctcttacttggagggtcaagctgccaaaga
gttcattgcctggttggttagaggtagaggttag
SEQ ID NO: 7 (nucleic acid sequence encoding the N-terminal extension NE-3 sequence
for Corynebacterium slutamicum)
gaagaacaggcagaa
SEQ ID NO: 8 (nucleic acid sequence encoding the modified TEV cleavage site for
Corynebacterium slutamicum)
gaaaacctgtacttccag
SEQ ID NO: 9 (nucleic acid sequence encoding the lirapeptide for Corynebacterium
slutamicum)
cacgcagaaggcacctttacctccgatgtgtcctcctacctggaaggccaggcagcaaaaga
attcattgcatggctggt tcgcggtcgcggttag
SEQ ID NO: 10 (nucleic acid sequence encoding the N-terminal extension NE-3 sequence
for Escherichia coli)
gaagaacaggcagaa
SEQ ID NO: 11 (nucleic acid sequence encoding the modified TEV cleavage site for
Escherichia coli)
gaaaacctgtacttccag
SEQ ID NO: 12 (nucleic acid sequence encoding the lirapeptide for Escherichia coli)
catgcggaaggcaccttcaccagcgatgttagcagctacctggagggtcaggcggcgaagga
atttatcgcgtggctggttcgtggccgtggttaa
SEQ ID NO: 13 (nucleic acid sequence encoding the N-terminal extension NE-3 sequence
for Bacillus subtilis)
gaagaacaagccgaa
SEQ ID NO: 14 (nucleic acid sequence encoding the modified TEV cleavage site for
Bacillus subtilis)
gagaacttgtacttccaa
SEQ ID NO: 15 (nucleic acid sequence encoding the lirapeptide for Bacillus subtilis)
cacgctgagggtacttttacctctgacgtgtcctcttacttggagggtcaagctgccaaaga
gttcattgcctggttggttagaggtagaggttag
SEQ ID NO: 16 (amino acid sequence of teriparatide)
SVSEIQLMHNLGKHLNSMERVEWLRKKLQDVHNF
SEQ ID NO: 17 (amino acid sequence of N-terminal extension sequence NE-1)
EEA
SEQ ID NO: 18 (nucleic acid sequence encoding the N-terminal extension NE-1 sequence)
gaggaagcg
SEQ ID NO: 19 (fusion protein comprising lirapeptide operably fused to N-terminal
extension sequence NE-3 and TEV cleavage site)
EEQAEENLYFQHAEGTFTSDVSSYLEGQAAKEFIAWLVRGRG
SEQ ID NO: 20 (fusion protein comprising lirapeptide operably fused to N-terminal
extension sequence NE-1 and TEV cleavage site)
EEAENLYFQHAEGTFTSDVSSYLEGQAAKEFIAWLVRGRG

### Definitions

Unless defined otherwise, all technical and scientific terms used herein have the same meaning as commonly understood by one of ordinary skill in the art to which the methods belong. Although any vectors, host cells, methods, and compositions similar or equivalent to those described herein can also be used in the practice or testing of the vectors, host cells, methods, and compositions, representative illustrations are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “host cell” includes an individual cell or cell culture which can be, or has been, a recipient for the subject of expression constructs. Host cells include progeny of a single host cell. The host cell for the purposes of this invention refers to any strain of Pichia pastoris, Saccharomyces cerevisiae, Corynebacterium glutamicum, Escherichia coli, and Bacillus subtilis which can be suitably used for the purposes of the invention.

The term “recombinant strain” or “recombinant host cell” refers to a host cell which has been transfected or transformed with the expression constructs or vectors of this invention.

The term “expression vector” or “expression construct” refers to any vector, plasmid or vehicle designed to enable the expression of an inserted nucleic acid sequence following transformation into the host.

The term “promoter” refers a DNA sequences that define where transcription of a gene begins. Promoter sequences are typically located directly upstream or at the 5′ end of the transcription initiation site. RNA polymerase and the necessary transcription factors bind to the promoter sequence and initiate transcription. Promoters can either be constitutive or inducible promoters. Constitutive promoters are the promoter which allows continual transcription of its associated genes as their expression is normally not conditioned by environmental and developmental factors. Constitutive promoters are very useful tools in genetic engineering because constitutive promoters drive gene expression under inducer-free conditions and often show better characteristics than commonly used inducible promoters. Inducible promoters are the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cells.

The term “expression” refers to the biological production of a product encoded by a coding sequence. In most cases, a DNA sequence, including the coding sequence, is transcribed to form a messenger-RNA (mRNA). The messenger-RNA is then translated to form a polypeptide product that has a relevant biological activity. Also, the process of expression may involve further processing steps to the RNA product of transcription, such as splicing to remove introns, and/or post-translational processing of a polypeptide product.

The term “modified nucleic acid” as used herein is used to refer to a nucleic acid encoding modified lirapeptide as represented by SEQ ID NO: 19 or 20 or a functionally equivalent variant thereof. Functional variant includes any nucleic acid having substantial or significant sequence identity or similarity to SEQ ID NO: 19 or 20, and which retains the biological activity of the protein.

The terms “polypeptide”, “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. “Amino acid” includes imino acid residues such as proline and hydroxyproline. The side chains may be in either the (R) or the (S) configuration.

The term “N-terminal extension” refers to a peptide or polypeptide sequence that is removably linked to the N-terminal amino acid of a desired polypeptide. In a preferred embodiment, the N-terminal extension comprises the amino acid sequence of SEQ ID NO: 1.

### DETAILED DESCRIPTION OF THE INVENTION

The present invention discloses N-terminal extensions, nucleic acids, vectors, and recombinant host cells for the efficient production of biologically active peptides such as lirapeptide.

The invention contemplates a multidimensional approach for achieving a high yield of recombinant lirapeptide in a host cell by providing an expression construct in which the nucleic acid encoding lirapeptide is operably fused to a modified gene sequence encoding TEV (Tobacco Etch Virus) cleavage site and an N-terminal extension (NE-3).

In one embodiment, the invention relates to the N-terminal extension sequence set forth in SEQ ID NO: 1 (NE-3). The invention relates to a process for the enhanced expression of recombinant therapeutic peptides by several folds using a short N-terminal extension sequence as set forth in SEQ ID NO: 1, in bacteria or yeast.

In another embodiment, nucleic acids encoding the N-terminal extension sequence set forth in SEQ ID NO: 1 (NE-3) are also covered within the scope of the invention.

Suitable host cell for the expression of a recombinant therapeutic peptide is selected from eukaryotic hosts, such as, but not limited to yeast which includes Pichia pastoris and Saccharomyces cerevisiae. Bacterial hosts, such as, but not limited to Corynebacterium glutamicum, Escherichia coli and Bacillus subtilis can also be used.

The term therapeutic peptide includes peptides, such as, but not limited to Lirapeptide, Teriparatide, Exenatide and the like.

Constitutive or inducible promoters known to a person skilled in the art can be used in the expression cassettes in one or more embodiments of this invention.

In another embodiment, the present invention provides expression cassettes comprising promoter, signal sequence, N-terminal extension (NE-3), gene encoding for Lirapeptide or Teriparatide, TEV cleavage site and terminator.

In an embodiment, the present invention provides N-terminal extension sequence as set forth in SEQ ID NO: 1 to enhance the expression of therapeutic peptide in yeast wherein the yeast is Pichia pastoris, Saccharomyces cerevisiae, Corynebacterium glutamicum, Escherichia coli and Bacillus subtilis.

The present invention also provides TEV cleavage site having the amino acid sequence set forth in SEQ ID NO: 2.

In an embodiment, the present invention provides enhanced expression of Lirapeptide as set forth in SEQ ID NO: 3 and Teriparatide as set forth in SEQ ID NO: 16 in bacteria or yeast using the N-terminal extension sequence as set forth in SEQ ID NO: 1.

Expression constructs known to person skilled in the art for expression of prokaryotic or eukaryotic proteins can be used in one or more embodiments of this invention.

In one embodiment, the present invention also provides an expression construct for the high-level expression of Lirapeptide which comprises of:
1. a modified gene sequence encoding the N-terminal extension sequence (NE3),
2. a modified gene sequence encoding TEV (Tobacco Etch Virus) cleavage site, and
3. a modified gene sequence encoding Lirapeptide.

In another embodiment, the present invention also provides an expression construct for the high-level expression of Lirapeptide which comprises of:
1. a gene sequence encoding the N-terminal extension sequence (NE-3) as set forth in SEQ ID NO: 4,
2. a gene sequence encoding TEV (Tobacco Etch Virus) cleavage site as set forth in SEQ ID NO: 5, and
3. a gene sequence encoding Lirapeptide as set forth in SEQ ID NO: 6.

In another embodiment, the present invention also provides a method for high level expression of Lirapeptide as set forth in SEQ ID NO: 3 in Pichia Pastoris which comprises:
1. construction of a recombinant vector (expression construct) comprising the gene sequences as set forth in SEQ ID NO: 4, 5 and 6,
2. transformation of the expression construct into Pichia Pastoris,
3. evaluation of the clone and selection thereof,
4. subjecting the selected clones to fermentation process,
5. isolation and purification of Lirapeptide, and
6. cleavage of the N-terminal extension sequence from the purified Lirapeptide.

In another embodiment, the present invention also provides an expression construct for the high-level expression of Lirapeptide which comprises of:
1. a gene sequence encoding the N-terminal extension sequence as set forth in SEQ ID NO: 7,
2. a gene sequence encoding TEV cleavage site as set forth in SEQ ID NO: 8, and
3. a gene sequence encoding Lirapeptide as set forth in SEQ ID NO: 9.

In an embodiment, the present invention also provides a method for high level expression of Lirapeptide as set forth in SEQ ID NO: 3 in Corynebacterium glutamicum which comprises:
1. construction of a recombinant vector (expression construct) comprising the gene sequences as set forth in SEQ ID NO: 7, 8 and 9,
2. transformation of the expression construct into Corynebacterium glutamicum,
3. evaluation of the clone and selection thereof,
4. subjecting the selected clones to fermentation process,
5. isolation and purification of Lirapeptide, and
6. cleavage of the N-terminal extension sequence from the purified Lirapeptide.

The present invention provides an expression construct for the high-level expression of Lirapeptide which comprises of:
1. a gene sequence encoding the N-terminal extension sequence as set forth in SEQ ID NO: 10,
2. a gene sequence encoding TEV cleavage site as set forth in SEQ ID NO: 11, and
3. a gene sequence encoding Lirapeptide as set forth in SEQ ID NO: 12.

In an embodiment, the present invention also provides a method for high-level expression of Lirapeptide as set forth in SEQ ID NO: 3 in Escherichia coli which comprises:
1. construction of a recombinant vector (expression construct) comprising the gene sequences as set forth in SEQ ID NO: 10, 11 and 12,
2. transformation of the expression construct into Escherichia coli,
3. evaluation of the clone and selection thereof,
4. subjecting the selected clones to fermentation process,
5. isolation and purification of Lirapeptide, and
6. cleavage of the N-terminal extension sequence from the purified Lirapeptide.

The present invention provides an expression construct for the high-level expression of Lirapeptide which comprises of:
1. a gene sequence encoding the N-terminal extension sequence as set forth in SEQ ID NO: 13,
2. a gene sequence encoding TEV cleavage site as set forth in SEQ ID NO: 14, and
3. a gene sequence encoding Lirapeptide as set forth in SEQ ID NO: 15.

In an embodiment, the present invention also provides a method for high-level expression of Lirapeptide as set forth in SEQ ID NO: 3 in Bacillus subtilis which comprises:
1. construction of a recombinant vector (expression construct) comprising the gene sequences as set forth in SEQ ID NO: 13, 14 and 15,
2. transformation of the expression construct into Bacillus subtilis,
3. evaluation of the clone and selection thereof,
4. subjecting the selected clones to fermentation process,
5. isolation and purification of Lirapeptide, and
6. cleavage of the N-terminal extension sequence from the purified Lirapeptide.

The present invention provides high level expression of Teriparatide as set forth in SEQ ID NO: 16 in Corynebacterium glutamicum using the expression construct comprising N-terminal extension sequence.

The present invention provides high level expression of Teriparatide as set forth in SEQ ID NO: 16 in Pichia pastoris using the expression construct comprising N-terminal extension sequence.

The present invention provides high level expression of Teriparatide as set forth in SEQ ID NO: 16 in Corynebacterium glutamicum which comprises the following steps:
1. construction of a recombinant vector (expression construct)
2. transformation of the expression construct into Corynebacterium glutamicum,
3. evaluation of the clone and selection thereof,
4. subjecting the selected clones to fermentation process,
5. isolation and purification of Teriparatide, and
6. cleavage of the N-terminal extension sequence from the purified Teriparatide,

The present invention also provides N-terminal extension sequence as set forth in SEQ ID NO: 1 to enhance expression of Teriparatide in Pichia pastoris which comprises the following steps:
1. construction of a recombinant vector (expression construct),
2. transformation of the constructed vector into Pichia pastoris,
3. evaluation of the clone and selection thereof,
4. subjecting the selected clones to fermentation process,
5. isolation and purification of Teriparatide, and
6. cleavage of the N-terminal extension sequence from the purified Teriparatide.

In another embodiment, the invention provides a modified lirapeptide, wherein the lirapeptide is operably fused to TEV (Tobacco Etch Virus) cleavage site and an N-terminal extension sequence (NE-3), and wherein the modified lirapeptide is as set forth in SEQ ID NO: 19.

In another embodiment, the invention provides a method for expressing lirapeptide using recombinant host cells of the present invention, wherein the fermentation process comprises:
a. culturing the recombinant host cells in BMGY media for about 24 hrs;
b. harvesting the recombinant host cells by centrifugation;
c. resuspending the recombinant host cells to an OD₆₀₀ nm of about 10 in BMMY medium;
d. incubating the host cells in a shaker incubator for about 24 hrs at 30° C.;
e. harvesting and purifying the culture supernatants to obtain lirapeptide.

Liraglutide, an analog of human GLP-1 and acts as a GLP-1 receptor agonist. Liraglutide is made by attaching a C-16 fatty acid (palmitic acid) with a glutamic acid spacer on the remaining lysine residue at position 26 of the peptide precursor (lirapeptide as set forth in SEQ ID NO: 3).

In another embodiment, the invention provides preparation of Liraglutide which comprises conjugation of lirapeptide produced as per the invention with palmityl glutamate derivative such as 1-methyl palmityl glutamic acid, using methods known in the art.

In another embodiment, the invention provides preparation of Liraglutide which comprises conjugation of lirapeptide produced as per the invention with palmityl glutamate derivatives, wherein derivatives are such as methyl (1-methyl palmityl glutamic acid), ethyl, propyl, prop-2-yl, butyl, but-2-yl, 2-methylprop-1-yl, 2-methyl-prop-2-yl (tert-butyl), hexyl and the like, using methods known in the art. This conjugation reaction is carried out in the presence of a coupling reagent. The coupling agent may be selected from the group of DIC/6-Cl-HOBt, DIC/HOBt, HBTU/HOBt/DIEA or DIC/Oxyma.

In another embodiment, the invention provides a method for preparation of liraglutide, said method comprising the steps of:
a. culturing the recombinant host cell of the present invention in a suitable culture medium to obtain lirapeptide;
b. converting lirapeptide to liraglutide, wherein the method comprises conjugation of lirapeptide obtained in step (a) with a palmityl glutamate derivative.

The above disclosure generally describes the present invention. A more complete understanding can be obtained by reference to the following specific examples. This example is described solely for the purposes of illustration and are not intended to limit the scope of the invention. Although specific terms have been employed herein, such terms are intended in a descriptive sense and not for purposes of limitation.

### EXAMPLES

### Example 1: Modified Nucleic Acid for Expression of Lirapeptide

Expression cassettes encoding for liraglutide precursor peptide was modified for optimum expression in Pichia pastoris, Corynebacterium glutamicum, Escherichia coli and Bacillus subtilis. The modified open reading frame comprising the nucleotide sequence encoding lirapeptide fused to a sequence encoding a TEV (Tobacco Etch Virus) cleavage site and a sequence encoding an N-terminal extension (NE-3 or NE-1). The preferred codons for expression in Pichia pastoris, Corynebacterium glutamicum, Escherichia coli and Bacillus subtilis have been used in place of rare codons.

As a control, an open reading frame comprising the nucleotide sequence encoding lirapeptide without any N-terminal extension was prepared.

For Expression in Pichia pastoris

For expression in Pichia Pastoris, the nucleotide sequence encoding lirapeptide, the nucleotide sequence encoding TEV cleavage site and the nucleotide sequence encoding the N-terminal extension was modified.

The nucleotide sequence encoding lirapeptide is represented by SEQ ID NO: 6. The nucleotide sequence TEV cleavage site is represented by SEQ ID NO: 5. The nucleotide sequence encoding N-terminal extension (NE-3) is represented by SEQ ID NO: 4.

This modified open reading frame has been artificially synthesized using the sequence for lirapeptide, sequence for TEV cleavage site and the sequence for N-terminal extension.

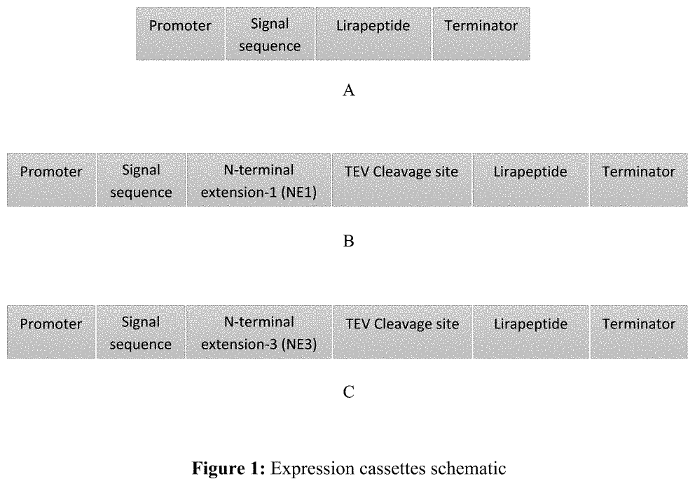

The modified open reading frame comprising DNA encoding Lirapeptide without N-terminal extension (FIG. 1A) and with N-terminal extension-1 (NE1) (GAGGAAGCG-FIG. 1B), N-terminal extension-3 (NE3) (GAAGAACAAGCCGAA-FIG. 1C), along with TEV (Tobacco Etch Virus) cleavage sequence (GAGAACTTGTACTTCCAA) and signal sequence cassettes are represented in FIG. 1.

The modified sequence encoding for the recombinant lirapeptide was cloned into pD912 expression vector (Atum, USA). The recombinant plasmid contains the open reading frame and a promoter.

The vector map of pD912 is represented in FIG. 1.

For Expression in Corynebacterium glutamicum

For expression in Corynebacterium glutamicum, the nucleotide sequence encoding lirapeptide, the nucleotide sequence encoding TEV cleavage site and the nucleotide sequence encoding the N-terminal extension was modified.

The nucleotide sequence encoding lirapeptide is represented by SEQ ID NO: 9. The nucleotide sequence TEV cleavage site is represented by SEQ ID NO: 8. The nucleotide sequence encoding N-terminal extension (NE-3) is represented by SEQ ID NO: 7.

This modified open reading frame has been artificially synthesized using Thermo Fisher Scientific technique utilizing the sequence for lirapeptide, sequence for TEV cleavage site and the sequence for N-terminal extension.

The modified sequence encoding for the recombinant lirapeptide was cloned into pD912 expression vector (Atum, USA). The recombinant plasmid contains the open reading frame and a promoter.

For Expression in Escherichia coli

For expression in Escherichia coli, the nucleotide sequence encoding lirapeptide, the nucleotide sequence encoding TEV cleavage site and the nucleotide sequence encoding the N-terminal extension was modified.

The nucleotide sequence encoding lirapeptide is represented by SEQ ID NO: 12. The nucleotide sequence TEV cleavage site is represented by SEQ ID NO: 11. The nucleotide sequence encoding N-terminal extension (NE-3) is represented by SEQ ID NO: 10.

This modified open reading frame has been artificially synthesized using the sequence for lirapeptide, sequence for TEV cleavage site and the sequence for N-terminal extension.

The modified sequence encoding for the recombinant lirapeptide was cloned into pD912 expression vector (Atum, USA). The recombinant plasmid contains the open reading frame and a promoter.

For Expression in Bacillus subtilis

For expression in Bacillus subtilis, the nucleotide sequence encoding lirapeptide, the nucleotide sequence encoding TEV cleavage site and the nucleotide sequence encoding the N-terminal extension was modified.

The nucleotide sequence encoding lirapeptide is represented by SEQ ID NO: 15. The nucleotide sequence TEV cleavage site is represented by SEQ ID NO: 14. The nucleotide sequence encoding N-terminal extension (NE-3) is represented by SEQ ID NO: 13.

This modified open reading frame has been artificially synthesized using the sequence for lirapeptide, sequence for TEV cleavage site and the sequence for N-terminal extension.

The modified sequence encoding for the recombinant lirapeptide was cloned into pD912 expression vector (Atum, USA). The recombinant plasmid contains the open reading frame and a promoter.

### Confirmation of Linearization of Plasmid DNA

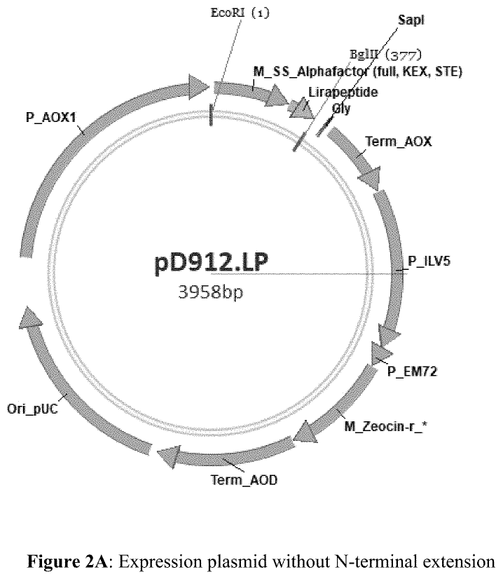

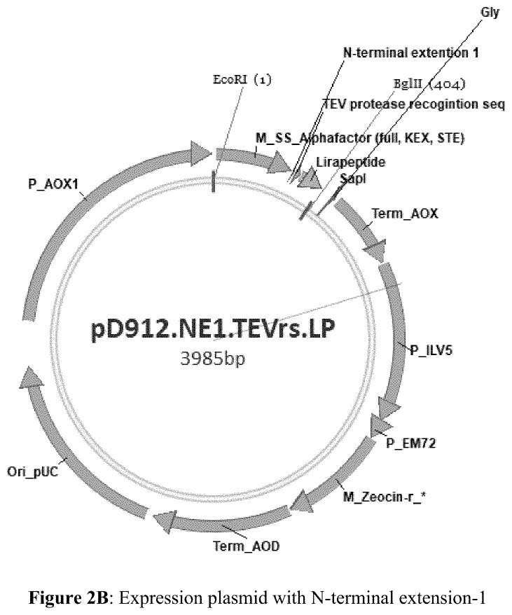

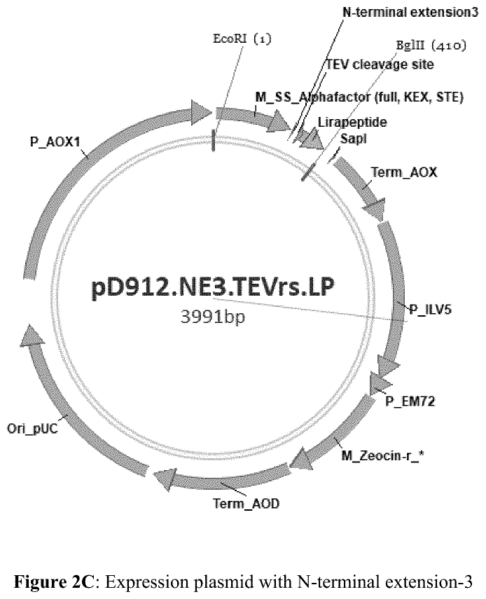

The synthetic DNA encoding Lirapeptide without N-terminal extension, N-terminal extension-1, N-terminal extension-3 and plasmid pD912 were digested with EcoRI and BglII restriction enzymes. The restriction digested fragments were ligated and transformed into Escherichia coli strain. The resultant plasmids, containing Lirapeptide expression cassettes, without N-terminal extension (FIG. 2A), N-terminal extension-1 (Figure-2B), N-terminal extension-3 (FIG.-2C) were sequenced to confirm Lirapeptide, N-Terminal extension, and TEV cleavage sequence. The sequence confirmed plasmid DNA's were linearized with Sac I enzyme.

### Example 2: Development of Recombinant Host Cell by Transformation with Recombinant Plasmids

Recombinant pD912 plasmids as described in foregoing example carrying the gene for liraglutide precursor peptide fused to signal peptides were used for development of recombinant hosts.

Pichia pastoris host cells (obtained from Atum, USA) were transformed using the plasmids by electroporation method.

The transformed cells were plated on YPD agar (Yeast Peptone Dextrose) plates containing 100 μg/ml zeocin. The transformed Pichia pastoris cells were grown in 20 ml BMGY media for 24 hrs. Cells were harvested by centrifugation and re-suspended to an OD₆₀₀ nm of 10 in 20 ml BMMY medium. The cell suspension was incubated in shaker incubator for 24 hrs at 30° C.

### Example 3: Analysis and Evaluation of Lirapeptide Expression

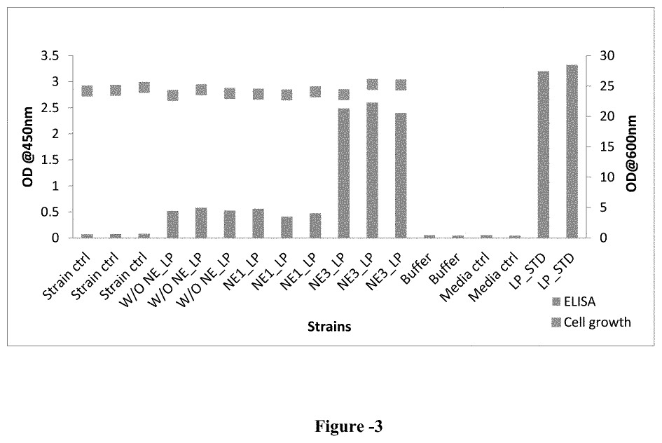

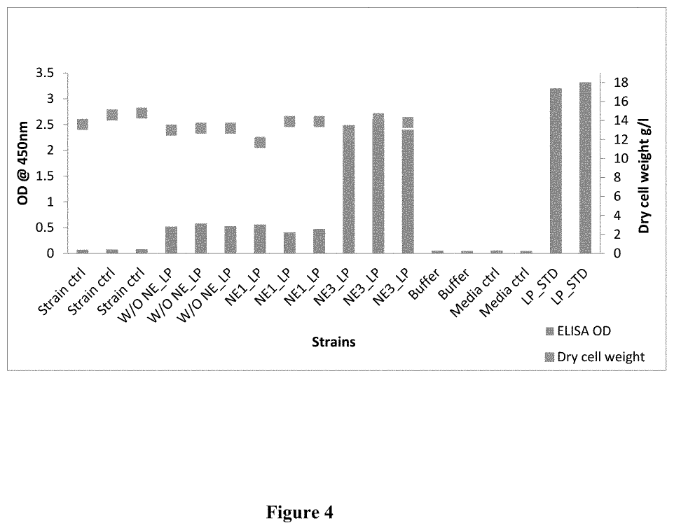

After 24 hours, the culture supernatants were harvested, purified and analysed for Lirapeptide expression by ELISA (FIG. 3) using monoclonal antibody specific to Lirapeptide. Further, the dry cell weight was measured using moisture analyser (FIG. 4).

Table 1 and Table 2 provides a comparison establishing the efficacy of the N-terminal extensions in improving the yield of lirapeptide.

TABLE 1
Different N-terminal extensions and Lirapeptide expression
compared to a control with no N-terminal extension

| | | LP expression | |
|---|---|---|---|
| Extension | Clones | (Percentage of control) | OD at 450 nm |
| None (control) | | 100 | 0.52 |
| EEA | 1 | 103 | 0.561 |
| EEA | 2 | 75 | 0.409 |
| EEA | 3 | 87 | 0.474 |
| EEQAE | 1 | 459 | 2.488 |
| EEQAE | 2 | 479 | 2.598 |
| EEQAE | 3 | 443 | 2.4 |

TABLE 2
Different N-terminal extensions and fold change in Lirapeptide
expression compared to control with no N-terminal extension

| | | Fold |
|---|---|---|
| Extension | Clones | difference |
| EEA | 1 | 1.0 |
| EEA | 2 | 0.8 |
| EEA | 3 | 0.9 |
| EEQAE | 1 | 4.6 |
| EEQAE | 2 | 4.8 |
| EEQAE | 3 | 4.4 |

The above data clearly shows that the N-terminal extension is able to improve the expression of lirapeptide by about 5-folds as compared to control and known N-terminal extensions.

1. An N-terminal extension comprising the amino acid sequence of SEQ ID NO: 1.

2. A nucleic acid encoding the N-terminal extension as claimed in claim 1.

3. The nucleic acid as claimed in claim 2, wherein the nucleic acid is selected from a group comprising SEQ ID NO: 4, SEQ ID NO: 7, SEQ ID NO: 10 and SEQ ID NO: 13.

4. A vector comprising the modified nucleic acid as claimed in 3.

5. The vector as claimed in claim 4, wherein the vector is pD912.

6. The vector as claimed in claim 4, wherein the vector comprises a modified TEV cleavage site selected from a group comprising SEQ ID NO: 5, SEQ ID NO: 8, SEQ ID NO: 11 and SEQ ID NO: 14.

7. A vector for recombinant expression of lirapeptide comprising:
a. a modified gene sequence encoding the N-terminal extension sequence (NE-3) selected from a group comprising SEQ ID NO: 4, SEQ ID NO: 7, SEQ ID NO: 10 and SEQ ID NO: 13;
b. a modified gene sequence encoding TEV (Tobacco Etch Virus) cleavage site selected from a group comprising SEQ ID NO: 5, SEQ ID NO: 8, SEQ ID NO: 11 and SEQ ID NO: 14; and
c. a modified gene sequence encoding Lirapeptide selected from a group comprising SEQ ID NO: 6, SEQ ID NO: 9, SEQ ID NO: 12 and SEQ ID NO: 15.

8. The vector as claimed in claim 7, wherein the vector is modified for high level expression of lirapeptide in Pichia pastoris comprising:
a. a modified gene sequence encoding the N-terminal extension sequence (NE-3) comprising the nucleotide sequence of SEQ ID NO: 4;
b. a modified gene sequence encoding TEV (Tobacco Etch Virus) cleavage site comprising the nucleotide sequence of SEQ ID NO: 5; and
c. a modified gene sequence encoding Lirapeptide comprising the nucleotide sequence of SEQ ID NO: 6.

9. The vector as claimed in claim 7, wherein the vector is modified for high level expression of lirapeptide in Corynebacterium glutamicum comprising:
a. a modified gene sequence encoding the N-terminal extension sequence (NE-3) comprising the nucleotide sequence of SEQ ID NO:7;
b. a modified gene sequence encoding TEV (Tobacco Etch Virus) cleavage site comprising the nucleotide sequence of SEQ ID NO: 8; and
c. a modified gene sequence encoding Lirapeptide comprising the nucleotide sequence of SEQ ID NO: 9.

10. The vector as claimed in claim 7, wherein the vector is modified for high level expression of lirapeptide in Escherichia coli comprising:
a. a modified gene sequence encoding the N-terminal extension sequence (NE-3) comprising the nucleotide sequence of SEQ ID NO:10;
b. a modified gene sequence encoding TEV (Tobacco Etch Virus) cleavage site comprising the nucleotide sequence of SEQ ID NO: 11; and
c. a modified gene sequence encoding Lirapeptide comprising the nucleotide sequence of SEQ ID NO: 12.

11. The vector as claimed in claim 7, wherein the vector is modified for high level expression of lirapeptide in Bacillus subtilis comprising:
a. a modified gene sequence encoding the N-terminal extension sequence (NE-3) comprising the nucleotide sequence of SEQ ID NO:13;
b. a modified gene sequence encoding TEV (Tobacco Etch Virus) cleavage site comprising the nucleotide sequence of SEQ ID NO: 14; and
c. a modified gene sequence encoding Lirapeptide comprising the nucleotide sequence of SEQ ID NO: 15.

12. A recombinant host cell comprising the vector as claimed in claim 7.

13. The recombinant host cell as claimed in claim 12, wherein the recombinant host cell is selected from a group comprising Pichia pastoris, Saccharomyces cerevisiae, Corynebacterium glutamicum, Escherichia coli and Bacillus subtilis.

14. A modified lirapeptide comprising the amino acid sequence of SEQ ID NO: 19, wherein the lirapeptide is operably fused to TEV (Tobacco Etch Virus) cleavage site and an N-terminal extension sequence (NE-3).

15. The method for expressing lirapeptide using recombinant host cells as claimed in claim 12, wherein the fermentation process comprises:
a. culturing the recombinant host cells in BMGY media for about 24 hrs;
b. harvesting the recombinant host cells by centrifugation;
c. resuspending the recombinant host cells to an OD₆₀₀ nm of about 10 in BMMY medium;
d. incubating the host cells in a shaker incubator for about 24 hrs at 30° C.; and
e. harvesting and purifying the culture supernatants to obtain lirapeptide.

16. A method for preparation of liraglutide, said method comprising the steps of:
c. culturing the recombinant host cell as claimed in claim 12 in a suitable culture medium to obtain lirapeptide;
d. converting lirapeptide to liraglutide, wherein the method comprises conjugation of lirapeptide obtained in step (a) with a palmityl glutamate derivative.


SEQUENCE LISTING (20 sequences)

SEQ ID NO: 1 - protein, 5 aa, ARTIFICIAL
  Amino acid sequence of N-terminal extension sequence NE-3
EEQAE

SEQ ID NO: 2 - protein, 6 aa, ARTIFICIAL
  Amino acid sequence of the modified TEV cleavage site
ENLYFQ

SEQ ID NO: 3 - protein, 31 aa, ARTIFICIAL
  Amino acid sequence of Lirapeptide
HAEGTFTSDVSSYLEGQAAKEFIAWLVRGRG

SEQ ID NO: 4 - DNA, 15 bp, ARTIFICIAL
  Nucleic acid sequence encoding the N-terminal extension NE-3 sequence for Pichia pastoris
gaagaacaagccgaa

SEQ ID NO: 5 - DNA, 18 bp, ARTIFICIAL
  Nucleic acid sequence encoding the modified TEV cleavage site for Pichia pastoris
gagaacttgtacttccaa

SEQ ID NO: 6 - DNA, 96 bp, ARTIFICIAL
  Nucleic acid sequence encoding the lirapeptide for Pichia pastoris
cacgctgagggtacttttacctctgacgtgtcctcttacttggagggtcaagctgccaaagagttcattgcctggttggttagaggtagaggttag

SEQ ID NO: 7 - DNA, 15 bp, ARTIFICIAL
  Nucleic acid sequence encoding the N-terminal extension NE-3 sequence for Corynebacterium glutamicum
gaagaacaggcagaa

SEQ ID NO: 8 - DNA, 18 bp, ARTIFICIAL
  Nucleic acid sequence encoding the modified TEV cleavage site for Corynebacterium glutamicum
gaaaacctgtacttccag

SEQ ID NO: 9 - DNA, 96 bp, ARTIFICIAL
  Nucleic acid sequence encoding the lirapeptide for Corynebacterium glutamicum
cacgcagaaggcacctttacctccgatgtgtcctcctacctggaaggccaggcagcaaaagaattcattgcatggctggttcgcggtcgcggttag

SEQ ID NO: 10 - DNA, 15 bp, ARTIFICIAL
  Nucleic acid sequence encoding the N-terminal extension NE-3 sequence for Escherichia coli
gaagaacaggcagaa

SEQ ID NO: 11 - DNA, 18 bp, ARTIFICIAL
  Nucleic acid sequence encoding the modified TEV cleavage site for Escherichia coli
gaaaacctgtacttccag

SEQ ID NO: 12 - DNA, 96 bp, ARTIFICIAL
  Nucleic acid sequence encoding the lirapeptide for Escherichia coli
catgcggaaggcaccttcaccagcgatgttagcagctacctggagggtcaggcggcgaaggaatttatcgcgtggctggttcgtggccgtggttaa

SEQ ID NO: 13 - DNA, 15 bp, ARTIFICIAL
  Nucleic acid sequence encoding the N-terminal extension NE-3 sequence for Bacillus subtilis
gaagaacaagccgaa

SEQ ID NO: 14 - DNA, 18 bp, ARTIFICIAL
  Nucleic acid sequence encoding the modified TEV cleavage site for Bacillus subtilis
gagaacttgtacttccaa

SEQ ID NO: 15 - DNA, 96 bp, ARTIFICIAL; identical to SEQ ID NO: 6
  Nucleic acid sequence encoding the lirapeptide for Bacillus subtilis

SEQ ID NO: 16 - protein, 34 aa, ARTIFICIAL
  Amino acid sequence of teriparatide
SVSEIQLMHNLGKHLNSMERVEWLRKKLQDVHNF

SEQ ID NO: 17 - protein, 3 aa, Artificial Sequence
  Amino acid sequence of N-terminal extension sequence NE-1
EEA

SEQ ID NO: 18 - DNA, 9 bp, Artificial Sequence
  Nucleic acid sequence encoding the N-terminal extension NE-1 sequence
gaggaagcg

SEQ ID NO: 19 - protein, 42 aa, Artificial Sequence
  Fusion protein comprising lirapeptide operably fused to N-terminal extension sequence NE-3 and TEV cleavage site
EEQAEENLYFQHAEGTFTSDVSSYLEGQAAKEFIAWLVRGRG

SEQ ID NO: 20 - protein, 40 aa, Artificial Sequence
  Fusion protein comprising lirapeptide operably fused to N-terminal extension sequence NE-1 and TEV cleavage site
EEAENLYFQHAEGTFTSDVSSYLEGQAAKEFIAWLVRGRG